(19) United States
(12) Patent Application Publication
(10) Pub. No.: US 2015/0339953 A1
(43) Pub. Date: Nov. 26, 2015

(54) GUIDANCE/SELF-LEARNING PROCESS FOR USING INHALERS

(71) Applicant: Fenil Shah, Houston, TX (US)

(72) Inventor: Fenil Shah, Houston, TX (US)

(21) Appl. No.: 14/284,693

(22) Filed: May 22, 2014

(51) Int. Cl. G09B 23/28; G09B 5/06; A61M 15/00

(52) U.S. Cl. CPC G09B 23/28; A61M 15/0065; G09B 5/065

(57) ABSTRACT

A self-learning system of providing guidance on using inhaler devices, provided using a portable or mobile computing device. Guidance comprises of multiple stages(modes), and provides a unique feature of real-time learning using a camera device. It provides simultaneous viewing of the instructional video and the patient's real-time video in real time. The said guidance is provided in step-by-step instructions in an incremental manner of increasing complexity. The steps of the guidance are linked together by an audio-visual indicator. The self-learning system of using inhaler devices has various features including action-focusing videos, a technique to superimpose an animation on the learner's real-time video and virtual placebo inhaler. The said system of using inhalers also provides guidance on learning, practicing/reinforcing the correct method of using inhalers and assists the patient in using the inhaler on a regular basis by logging, analyzing and alerting about the time and dosage of the inhaler.

### REFERENCES CITED

US Patent Documents
U.S. Pat. No. 7,095,388B2 8/2006 Truxa et al
U.S. Pat. No. 6,068,559 5/2000 Lubell et al

### CROSS-REFERENCE TO RELATED APPLICATIONS

This Patent application claims the benefit under 35 U.S.C. §119(e) of U.S. Provisional Patent Application No. 61/826,076 filed on 22 May 2013, entitled, “Guidance/Self-Learning Process for Using Inhalers,” the contents and teachings of which are hereby incorporated by reference in entirety

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### TECHNICAL FIELD

The invention relates to remote medical guidance. Remote medical guidance basically refers to supervision or guidance of a medical procedure (in this case, how to use inhalers correctly) that is provided from a remote location. This application falls in the realm of real-time guidance.

More specifically, this innovation relates to providing real-time guidance to patients/related medical professionals on how to use an inhaler correctly. It covers all the major aspects of using inhaler devices-right from learning the correct technique of using inhalers to practicing/reinforcing the method of using inhalers with this innovation on a regular basis; all this being provided using portable/mobile computing devices.

### BACKGROUND OF THE INVENTION

In the field of medical procedures and more specifically for patients using medical devices such as inhalers used in Asthma, COPD, Cold, Cough, Wheezing and Allergies the knowledge of the procedure and technique is critical to the improvement of the patient's condition.

Various research studies have time and again established this fact. However, despite the reality, various factors impede the transmission of this knowledge to patients. The factors, amongst all things mainly include; (i) Unwillingness of patients to acknowledge that their technique is not correct, (ii) Busy schedules of health-care providers and professionals and (iii) Health-care providers are paid short or none for patient education about inhaler usage. (iv) Some of the health-care providers and professionals themselves are not aware of the correct technique. (v) Available videos are do not cover all the aspects of using inhaler devices and have various shortcomings.

A study from Respiratory Medicine (2007) 101, 2395-2401 shows that Verbally Trained COPD patients have better pre and post Peak Inhalation Flows (PIF) as compared to non-trained patients.

Another study that was conducted to determine the effect of training on inhaler technique and the quality of life on COPD patients by the Department of Medical Nursing, Erciyes University in which patients (study cases) were given training, demonstration and a leaflet; a follow-up was done after 3 months. The results indicate that the training provided decreased attack frequency, dyspnea and improved quality of life.

A research from Respiratory Care, September 2005 Vol 50 No 10 shows that out of the $25 billion spent on inhalers, $10 billion is wasted due to poor technique of using inhalers.

An Observational Study of 3811 patients of handling inhaler devices was conducted at 1 Aerosol Med 2003 indicated that over 50% of patients committed at least one error and over 12% of the patients committed a critical error while real life handling of the inhaler device. This again highlights the importance of providing guidance and reinforcing the correct usage of inhaler.

A research conducted by the Department of Medicine, University of Barcelona, Spain to evaluate the correctness of inhalation technique in a nationwide sample of patients and medical personnel in the use of Metered Dose Inhalers suggested that only 9% of patients, 15% of nurses and 28% of physicians showed a correct inhalation technique. This study shows the need of substantial changes in educational efforts.

Also, statistics from Chest 1994; 105:111-116 show that medical personnel's' knowledge of and ability to use inhaling devices is not adequate. The study suggests that only 65% of Respiratory Therapists, 39% Registered Nurses and 48% of Staff Physicians had the knowledge and ability of using Inhaler devices correctly.

In fact, it is quoted by Fink J B in Respir Care 2005 October that: “Management of Chronic disease is 10% medication and 90% education.”

Assuming even if the health-care professionals are experts in the technique, which a bunch of them really are, they cannot find time for each patient, to teach them the technique, the first time and every-time. They face constraints in educating patients due to lack of time, lack of training and due to no response from the patients.

The technique of using inhalers not only involves how to use the device but also how to inhale with the device such that the maximum amount of medicine is deposited in the lungs. This avoids wastage of medicine, which is mostly the case when inhalation of the drug does not happen as required.

Several studies have been done, which concluded that incorrect technique led to patients admitted for emergency thus adding a huge cost and administration burden to healthcare management.

Improper Inhaler technique is a serious problem. There are different types of inhaler devices available in the market. Also each device requires a different inhalation technique. The patient's ability to learn the technique varies. The teaching provided also varies. Lack of clear instructions can pose a problem to the patient.

The skills of using an inhaler device correctly can't be learned easily from written or verbal instructions. The written and/or verbal instructions have access problems. They have to be brought from the healthcare providers. Also, at the time of using the inhaler they are not handy. The instructions available with the healthcare providers are complex, lengthy, tedious and boring. Also they are not appealing to younger patients. Also, there is no self assessment in real-time. There is poor retention which leads to technique deterioration over time.

At times, a patient is required to use more than one type of inhaler. Different inhalers require different breathing patterns which often confuse the patient and results in wrong usage of the inhaler leading to wastage and thus preventing him from deriving maximum benefit of the medicine. It has also been observed that without continuous reinforcement, even patients who are taught the technique appropriately and who have been able to demonstrate it correctly then, can hardly remember everything. And that too, many-a-times immediately after a week of them being trained

Similarly, there are several such instances in the medical as well as other fields where procedures and techniques need to be taught to individuals in a systematic and consistent manner and reinforced with repeated and regular trainings.

The videos that are already available online (for instance, youtube videos) are unable to cover all the aspects of using inhaler devices. The basics of the correct breathing technique while using inhalers are not taught in most of the videos. Also, some videos show the procedure (steps) of using inhalers in a quick succession which makes it difficult for the patient to remember all the steps. Also, other aspects of using inhalers like priming, cleaning the inhaler device and rinsing of the mouth are not shown in the already available videos. More importantly, there is no real-time assessment of the technique. Even after watching the whole video there is no guarantee that the patient/related health professional will remember all the steps and use the inhaler device correctly.

Another major shortcoming is that all the verbal/written/video instructions are not handy on a regular basis while using the inhalers. The patient will not take the trouble to read through or watch the video every time he uses the inhaler.

Thus, there is a need for a breakthrough technology to overcome the above mentioned problems.

### SUMMARY OF THE INVENTION

It is an object of the present innovation to solve the problems described above. This innovation provides a simulation technology. It can be accessed using internet or mobile computing devices. Thus, it is accessible anytime anywhere at the patient's/related health professional's convenience. This innovation has a simulator ‘instructor’ that walks the patient/related health professional through the complex steps of inhalation. It is an easily available, inexpensive and interactive tool for the patient/related health professional. The innovation provides practice of the inhalation technique using a webcam/camera device. This helps in real-time viewing and thus provides real-time feedback which enhances the learning and self-efficacy. It can also be used on a regular basis for reminding, providing real-time assistance, logging and analyzing the usage of the inhaler.

The current process-innovation provides a suite of guidance and learning on how to use inhalers effectively to achieve the objectives mentioned above. This guidance and learning can be delivered anytime-anywhere through the use of any kind of computing device including wearable, hand-held, table-top, implantable, etc

Thus the number of ER visits, the cost of the inhaler device and the inhaler medicines, the time and cost of the health-care providers can all be reduced by using the self-learning/practicing procedure for using Inhaler devices. The self-learning procedure leads to cost saving as well as time saving.

The present approach “Guidance/Self-Learning Process for Using Inhalers” relates to an incremental learning approach in a specific sequence that includes (i) how to use any type of inhaler device, (ii) how to breathe (Inhale) through the device and optimize the breathing technique,) what mistakes to avoid (v) reinforcing these in real-time while using the inhaler. (v) guidance on processes like priming, cleaning and rinsing (vi) reminding and alerting about the inhaler dose to be taken on a regular basis. (vii) logging usage by the patient for analysis and finally (viii) capturing weather parameters and spatial location of the patient. This may be delivered on any and all computing and display devices.

Even though, an estimated 70% of Asthma and COPD patients are not able to use inhalers correctly, health-care professionals including doctors, nurses, and respiratory therapists are not able to reach to all the patients and train them diligently. And even if they are able to provide them the much needed training, there is no way and almost impossible to do it systematically and teach them all aspects of using the inhaler that will result into maximization of medicine intake. Besides, this entire process will be so much manual. Moreover, it has also been observed that patients, who are trained by health-care professionals during their visit to their office, tend to forget the key steps after, as little as a week.

In that context, there is a need to provide a method/process that automatically steers the patient's learning through the entire process without the manual intervention of the health-care professional, such as an automated self-learning process. This has several advantages amongst many others, such as saving the time of the health-care professional, providing repeated training as many times as the patient needs, being self-paced according to her/his time and convenience.

The innovation talks about automating this learning process, which is divided into multiple stages (2 or more stages) with each of the stage connected pedagogically through a common interactive audio-visual indicator.

While the innovation revolves around this concept, there are other aspects of the innovation that are also unique. The various stages, mentioned in the previous paragraph (and described under claim 1), each provide a unique experience to the patient's learning process.

By continuous usage of this innovation, the patient is able to ensure that the correct technique at all stages of inhaler usage, is communicated directly or indirectly to her/him through a set of automated applications. Increased compliance to usage of inhalers not only helps the patients with enhanced medicine intake, but also helps health-care providers, pharmaceutical companies by increasing efficacy and saving time.

One more point to be noted is that there are several patients who take inhaler puffs on a daily/regular basis and are unable to remember the time and amount of dosage to be taken. Also, owing to things like age and busy lifestyles some patients often forget to take their inhaler dose. Thus, they are unable to derive maximum benefit of the inhaler medicine and thus the treatment.

This proves that there is a need to provide a method/system that automatically reminds the patient about the time and the required inhaler dose. This will have several advantages. It will be an automated system. Thus, there will be no need for a parent or caregiver to remind the patient of his daily/regular dose of the inhaler. Also, since the patient will not skip his doses, he will derive maximum benefit from the inhaler and his treatment overall.

This is one unique aspect of the innovation. It consists of multiple stages (2 or more stages/modes) that teach and remind the patient about his regular/daily dose of the inhaler. Hence, if the patient has forgotten the correct technique of using the inhaler or the technique of any other related processes (like priming, cleaning and rinsing), he can use the audio or audio-visuals provided in this feature to take his inhaler medication.

Further another unique feature of this innovation is that it allows logging and recording of real-time practice sessions. This can be used for analysis by parent/caregiver/health-professional.

The unique features of this aspect of the innovation as mentioned in the above paragraphs (described in claim 1) helps make a patient's life easier. By ensuring that the patient has taken his inhaler dose correctly on a regular/daily basis and that he does not miss his dose, helps him gain maximum benefit from the treatment.

### BRIEF DESCRIPTION OF DRAWINGS

FIG. 1: It shows a representation of Self-learning process with multiple stages of learning;

FIG. 2: It shows a representation of Learning and practicing the steps;

FIG. 3a: Shows an example of Sample Stage 1—How to Inhale—The instructor demonstrating how to breathe in and breathe out;

FIG. 3b: It shows an example of Sample Stage 1—How to Inhale—The instructor demonstrating using a retractable pen on when to press the inhaler;

FIG. 4: It shows a representation of Sample Stage 2—How to Use Inhaler—Instructor demonstrating how to load a dose for a Metered Dose Inhaler (MDI) device;

FIG. 5a: It shows a representation of Samples Stage 3—Inhaler Dos and Don'ts—Dos and Don'ts arranged in the form of Questions;

FIG. 5b: It shows a representation of Sample Stage 3—Inhaler Dos and Don'ts—Animation explaining what happens if the patient does not shake the inhaler correctly;

FIG. 6: It shows a representation of Sample Stage 4—Assist Me with Inhaler—Choosing the inhaler used by the patient;

FIG. 7a: It shows a representation of Sample Stage 1—How to Inhale—Learning process begins with the first step of Breathing in and out;

FIG. 7b: It shows a representation of Sample Stage 1—How to Inhale—Sample Step 2 progresses to the addition of a Breath Bar;

FIG. 7c: It shows a representation of Sample Stage 1—How to Inhale—Sample Step 3 is added with a Breath Bar Audio Tune to the Breath Bar;

FIG. 7d: It shows a representation of Sample Stage 1—How to Inhale—Sample Step 4 advances to the Press Inhaler Indicator;

FIG. 7e: It shows a representation of Sample Stage 1—How to Inhale—Sample Step 5 introduces the patient on how to hold the breath once the basic procedure is done;

FIG. 7f: It shows a representation of using the Practice mode. The patient can then practice all these steps one-by-one by looking at himself/herself and verify if s/he is doing it correctly;

FIG. 8: It shows a representation of the Sample Breath Bar—The Visual Component;

FIG. 9a: It shows a representation of sample Cells indicating Slow and Deep Breath;

FIG. 9b: It shows a representation of sample Cells indicating Quick and Deep Breath;

FIG. 10: It shows a representation of sample Press Inhaler Indicator;

FIG. 11: It shows a representation of Sample Breath Bars for different types of inhalers;

FIG. 12: It shows an instance of each step being represented by a unique icon;

FIG. 13: It shows a representation of Example 2 of Superimposition of transparent imaging on the patient's real-time video;

### DETAILED DESCRIPTION OF THE PREFERRED EMBODIMENTS

The preferred embodiments of the present invention will be described herein with reference to the drawings in Figures. Those ordinarily skilled in the art will appreciate that the elements in the figures are illustrated for simplicity and clarity and are not necessarily drawn to scale.

As used in the specifications and claims, the singular forms “a”, “an” and “the” include plural references unless the context clearly indicates otherwise.

### TERMS USED

Inhaler: Inhaler is a medical device (of many different types) used by patients suffering from various ailments such as Asthma, COPD, Allergies, etc.

Stage/Mode: Stage is a logical division (or milestone) of a set of instructions or learning in the entire learning process on how to use an inhaler. Each stage may be delivered through software applications that can be delivered using computing devices such as table-tops, lap-tops, hand-held devices, wearable, implantable, etc.

Step: Step is a division of a Stage into one unique action and arranged in a sequence with other steps. A group of steps may depict one Stage.

Breath Bar: The Breath Bar is an animated representation (or an audio-visual indicator) that represents inhalation and exhalation of the human breathing process.

Breath Bar Audio/Breath Bar Audio Tone: The Breath Bar Audio Tone helps the learner to follow the breathing pattern for each inhaler by a sound pattern vis-à-vis the visual component the Breath Bar animation.

Press Inhaler Indicator: The Press Inhaler Indictor appears on the Breath Bar for inhalers where the patient needs to actuate or press the inhaler canister or button to release the medicine.

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The self-learning process of using an inhaler device is such that the patient learns and practices at her/his own pace. The entire process is in multiple stages of guidance in a sequence to provide learning using a camera on a computing device.

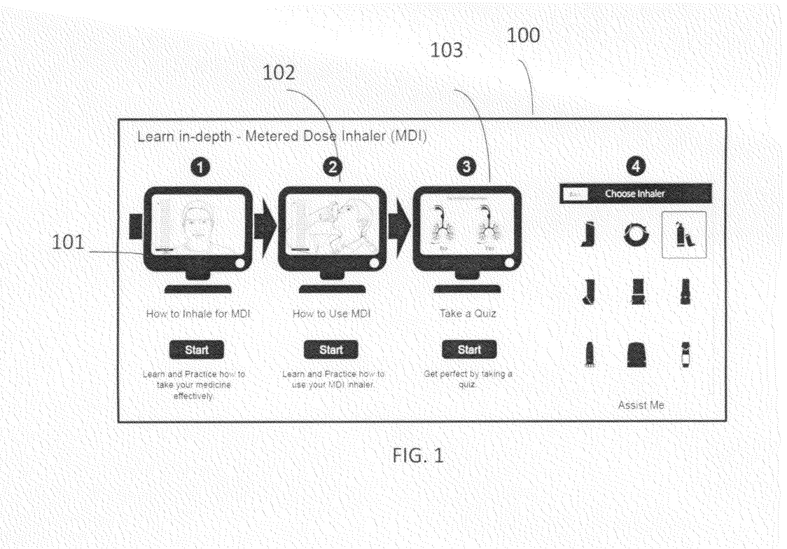

The self-learning process of using an inhaler device is unique due to its inclusion of multiple stages (2 or more stages) or guidance arranged in a manner such that the patient is able to learn and practice herself/himself without the intervention of a real trainer. Referring to FIG. 1, an introductory screen 100 includes available instances 101, 102, 103 and 104 including usage of an Inhaler 101, technique for using Inhaler the 102, a quiz 103 for evaluating competency with the chosen Inhaler and an application (104) for setting a reminder which allows to chose from a wide range of inhalers. (FIG. 1).

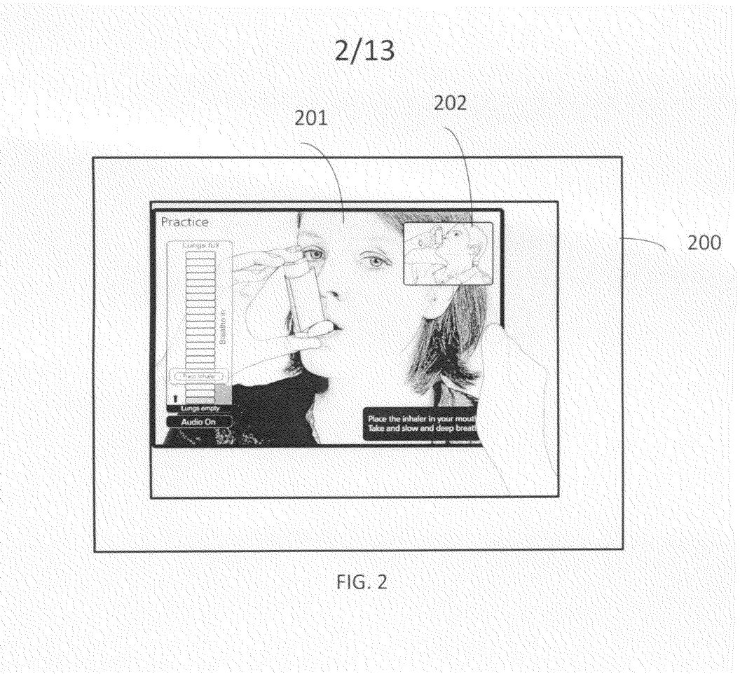

This self-learning process allows the patient to learn and understand and then practice by seeing herself/himself, in a way that is not even possible with a real trainer. Each instance has a plurality of guidance stages (stages), which each include various multimedia interactions depending on the stage. The user generally iterates through the defined stages and obtains a feedback result indicative of the success of each stage. Upon completion of each stage, the user may revert to a previously rendered stage or advancing to a successive stage based on user feedback of success of the stage. In this manner, a user may iteratively advance through the plurality of stages. For example, referring to FIG. 2, the stages including an inhalation stage having simultaneous rendering of a real-time user video stream 201 alongside an instructional video stream 202 depicting correct usage of the inhaler that can be viewed on a mobile/computing device 200. The real-time videos stream 201 results from an adjacent camera showing the user their movements in conjunction with the preferred actions in the instructional video stream 202. (FIG. 2)

This is a process where the patient learns and practices on her/his own pace. The entire process is in multiple sequences with the final stage providing real-time assistance during the actual usage of the selected inhaler to reinforce what was learned in the previous stages.

### Description of an Instance:

The inhaler self-guidance on how to use different types of inhalers consists of 3 Stages of guidance followed by 1 Stage of real-time assistance. This includes, First Stage of How to Inhale followed by Second Stage of How to Use a selected Inhaler followed by Third Stage of Dos and Don'ts of using the selected Inhaler and ends with the final and fourth stage of real-time assistance titled as Assist Me with the Inhaler. The 4 stages is only an instance of the description of multiple stages and is not really limited to this number.

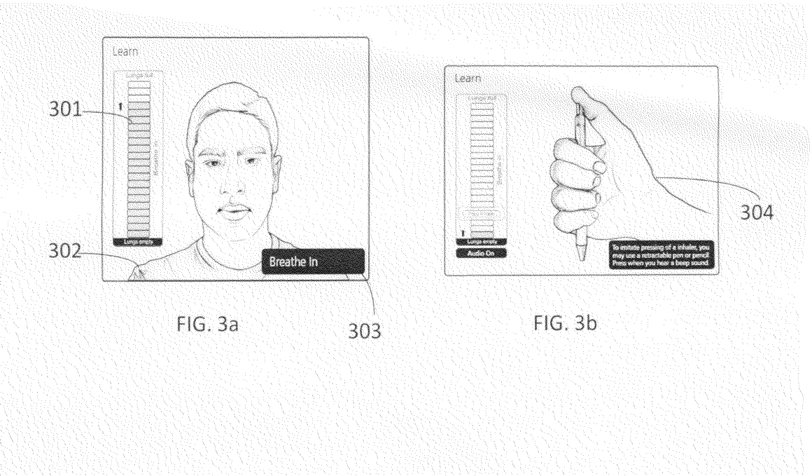

First Stage (How to Inhale)—(FIGS. 3a & 3b)
The first stage starts with explaining the patient on how to inhale (breathe-in) depending on what inhaler s/he chooses.
As shown in FIG. 3a the instructor 302 on the display/screen 300 demonstrates how to inhale (breathe-in) with the help of a breath-bar 301. Also this instance displays text 303 on the screen 300 which provides written description of the actions being performed by the instructor 302.
In another instance in FIG. 3b another technique of breathing with an inhaler is demonstrated using a pen 304. In this instance also, there is text used to display the actions being performed by the instructor.
This is the stage where the patient begins and sets the first stepping stone for the process.
All concepts learned and practiced at this stage are carried forward to the next stage and subsequence stages.

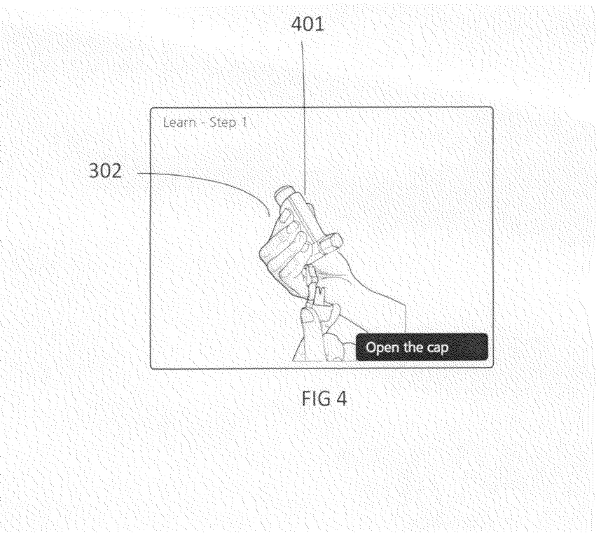

Second Stage (How to Use the Inhaler)—(FIG. 4)
Once the patient is through with this training, he is guided to the second stage which explains on how to use the inhaler device.
This guidance is provided with videos that are precise and arranged step-by-step.
Each instruction is provided by a separate step, so that multiple instructions are not provided to the patient, all at one go. This avoids a bunch of confusion compared to ordinary videos.
For example referring to FIG. 4 wherein the instructor 302 demonstrates the correct usage of the MDI 401.

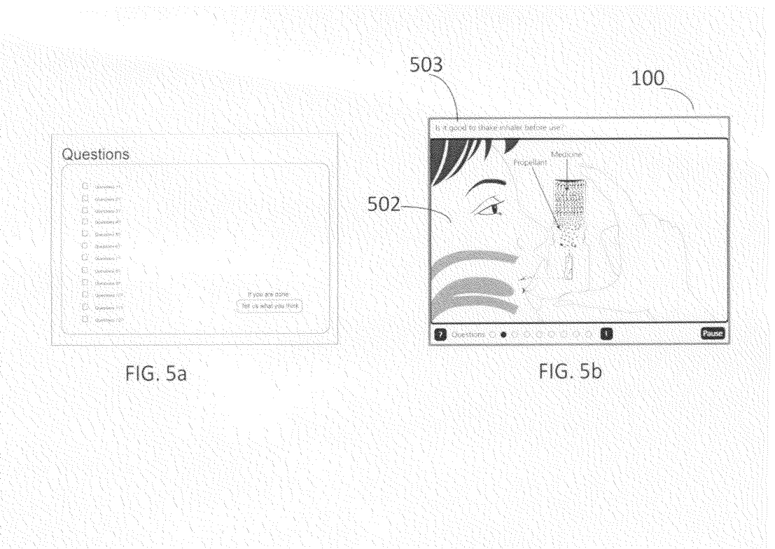

Third Stage (Inhaler Dos and Don'ts)—(FIGS. 5a & 5b)
The third stage navigates the patient through an interactive query guide that demonstrates
What mistakes to avoid while using the selected inhaler and
What happens internally within her/his body if s/he does something wrong or even something right
The more a patient is able to understand what happens when s/he does something right or wrong, the better s/he is able to remember the instructions and the better is her/his compliance.
Further, the Dos and Don'ts stage is arranged in the form of a Quiz. This makes the patient seek answers to questions that are part of the Dos and Don'ts list, encouraging constructive learning.
As shown in FIG. 5a the patient/related health professional is shown a list of questions regarding the usage of the inhaler device.
In another instance, the patient/related health professional is shown an audio-visual 502 along with the question 503 on the screen of the mobile computing device 100.

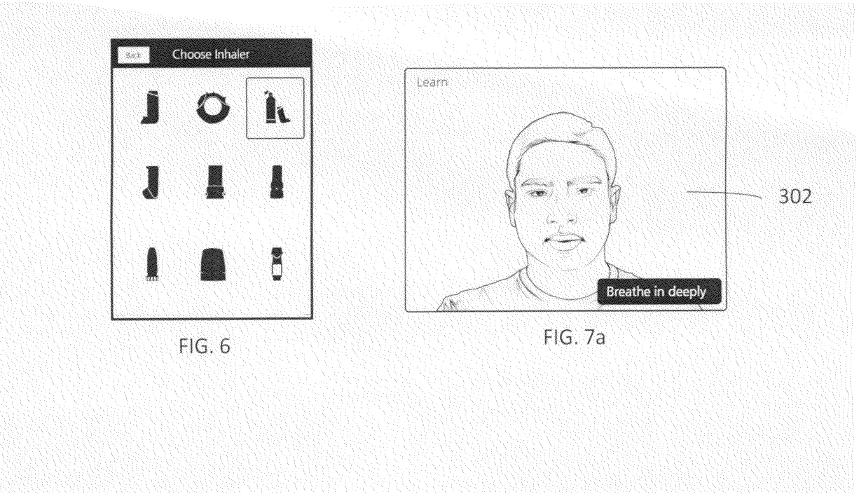

Fourth Stage (Assist Me with the Inhaler)—(FIG. 6)
The entire learning exercise goes to a naught if the patient, even after undergoing training, does not use the inhaler correctly. To avoid this, the process is concluded with a final and fourth stage.
This is a hand-held device application that reinforces the steps and techniques introduced in the previous 3 stages.
Most importantly, this reinforcement is provided to the patient right at the time of using the inhaler.
Just so that the patient does not forget to use the application, it is accompanied with a dosage alarm.
Thus, the patient can not only use the inhaler but also keep herself/himself reminded of the steps s/he had learned during the previous 3 stages.
An instance of the same is shown in FIG. 6, wherein the screen prompts the patient to select the type of the inhaler device to be used.

Concluding the last stage with real-time assistance makes sure that the patient is reliant on a mechanism that consistently does not allow her/him to falter on the technique, even while s/he is using the inhaler. Consequently, this leads to better medicine intake and thus improvement of her/his condition.

Guidance progresses from simple-to-do actions and then incrementally and gradually advances into more complex actions. All this while, the patient is both learning as well as practicing along with the instructor at her/his own pace, without having to worry how much time is going by, because the training is happening virtual and online.

### Description of an Instance:

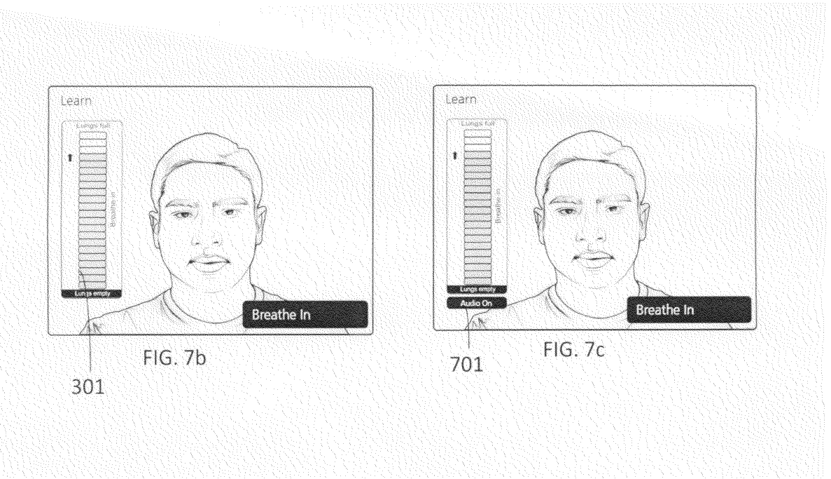

Stage 1 or How to Inhale consists of multiple-steps that guide the patient on how to inhale using an Inhaler device to maximize medicine intake to her/his lungs. This guidance begins the learning process with breathing-in and breathing-out as the First Step. The patient needs to follow the instructor 302 and sync her/his breath with the instructor 302 as shown in FIG. 7a.

As the next step (Step 2) in Stage 2 of learning, the patient now needs to sync her/his breath with a unique Breath Bar animation 301 as shown in FIG. 7b.

The Breath Bar helps the user to understand the breath pattern to use with that inhaler and breathe accordingly. The sequence goes like this—Instructor+Breath Bar. (FIG. 7b)

At Step 3, the instance of which is shown in FIG. 7c, the Breath Bar animation 301 movement is accompanied with a Breath Bar audio tone 701. This adds sound to breathe-in and breathe-out exercise. Why it is used, will be more clear in step 6. The sequence goes like this—Instructor+Breath Bar+Audio Tone. (FIG. 7c)

At Step 4, the Breath Bar animation 301 and audio tone 701 are accompanied with a “Press Inhaler” Indicator 702 and Beep Tone. This tone helps the patient to understand when to press the inhaler while breathing-in. The sequence goes like this—Instructor+Breath Bar+Audio Tone+Press Inhaler Indicator and Beep Tone. (FIG. 7d)

While using the inhaler the patient has to allow the medicine to settle in her/his lungs and therefore needs to hold her/his breath for a maximum of 10 seconds. Step 5 accompanies a timer 703 to help the learner understand how and how much to hold her/his breath after breathing in. Once done, he will need to breathe out. This entire pattern is shown visually in a sequence that goes like this—Instructor+Breath Bar+Audio Tone+Press Inhaler Indicator and Beep Tone+Timer (to hold breath). (FIG. 7e)

The final step, Step 6 sequence goes like this—Instructor+Audio Tone+Press Inhaler Beep Tone+Timer (to hold breath). All visual parts are cleared from the screen in this step. This is to make sure that the user remembers the correct synchronization of the breath pattern with the release of the inhaler by just listening to the Breath Bar Audio Tone and the Press Inhaler Beep Tone. This way the learning is rooted deep into the learner's sub-conscious memory if s/he continues to repeat this a few times until s/he does not need to look at the visual components, anymore.

Depending on the type of inhaler to learn, some of these stages may not apply. For instance, Stage 5 is applicable only for Metered Dose Inhaler and not for say a Diskus. Further, to enable the patient to practice each of these steps, the Stage 1 and Stage 2 applications provide an innovative method to enable the patient to see herself/himself doing the steps side-by-side in real-time along with the instructor 302. As shown in FIG. 7f, the patient can view his real-time video 201 while practicing along with instructional video 202 on the screen of the mobile computing device 200. The entire training process is thus virtual and online and does not require a real human instructor to be present while the training is on.

The multiple stages (2 or more stages) of guidance as are logically linked through a common learning aid in the form of an audio-visual indicator. The learning aid or audio-visual indicator may differ depending on what type of guidance is being provided and what needs to be remembered by the learner across all stages.

An example of a common learning aid or the audio-visual indicator used in guidance on how to use inhalers has been described in detailed, here. One of the most critical steps to perform while using an inhaler is performing the correct breathing pattern for that inhaler and when to release the inhaler medicine. This becomes extremely essential to remember at all stages of the learning process as well as during actual usage. Hence, the learner has to be repeatedly instructed about this step at all stages and therefore be provided a common link (or an audio-visual cue) for better retention of learning and/or representation. The Breath Bar and the Breath Tone Audio are used as this audio-visual cue.

The Breath Bar is an audio-visual indicator that represents inhalation and exhalation of the human breathing process. The Bottom-to-Top animated movement represents breathing-in and the Top-to-Bottom animated movement represents breathing out.

The Breath Bar is aimed at providing ‘Optimized Breathing Technique’. It basically assists the patient in syncing his breathing to its own pattern. By doing so, it assists the patient in understanding how deeply to breathe and also the pace of breathing (fast or slow) depending on the type of inhaler device chosen. This technique once understood by the patient will in-turn lead to maximum benefit of using the inhaler device by maximum deposition of the drug or medicine in the lungs. Thus, it optimizes the breathing pattern of the patient to provide maximum benefit of the inhaler medicine.

Patients just have to follow these movements (patterns) of the breath bar to sync their own breathing pattern, thus helping them to understand how to breathe correctly, to improve medicine intake. (See components of the Breath Bar). Additionally, the Breath Bar animation is also accompanied with an Audio Tone that further represents the said pattern.

### Components of the Breath Bar

Visual Component

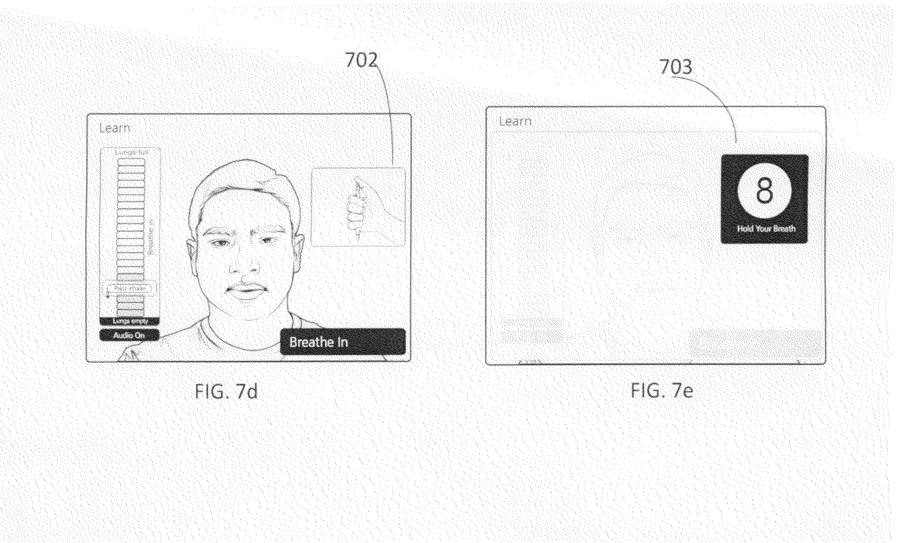

(An instance of the breath bar as shown in FIG. 8): Visually the breath bar is made up of (i) Lungs Capacity Indicator (LCI) panels 801, (ii) Individual colored cells or blocks 802, (iii) moving arrow 803, (iv) Press Inhaler Indicator (PII) 804 and (v) Text Labels 805.

### (i) Lungs Capacity Indicator Panel

These panels are placed horizontally at the top and bottom ends of the breath bar. They gradually glow Lungs Full or Lungs Empty when the moving arrow approaches them. The panels indicate that lungs are full or empty of air when the Breath Bar (BB) fills-up (or empties out) [or the animation arrow touches them].
These panels are common for all inhalers.

### (ii) Colored Cells or Blocks

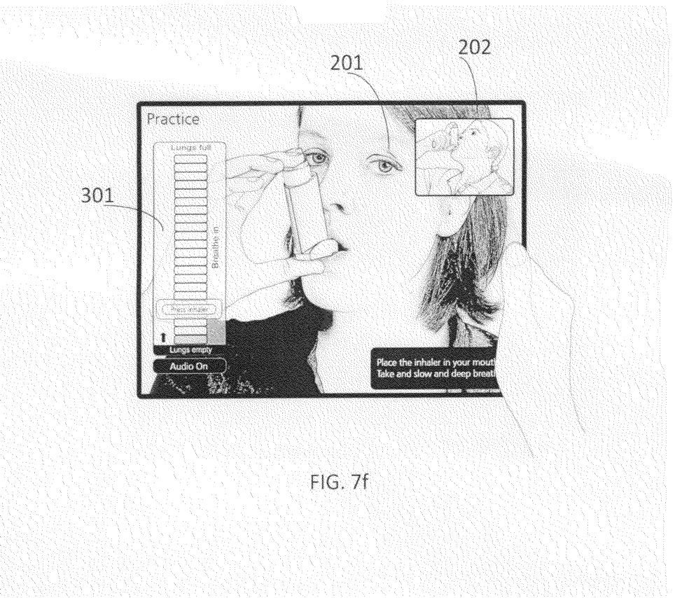

The Breath Bar is primarily made of colored cell/blocks placed one above the other in a vertical format. The blocks are color coded from red to amber to green from bottom to the top respectively. The red color appears at the bottom which denotes the lungs have very little air in them and the green color appears at the top indicating that the lungs are filled to capacity with air. The breath in/out movements is denoted by filling or emptying of the colors within grayed out blocks.
Note: The color codes may change. However, the basic concept of Lungs filling up or emptying out shall always be represented using the Breath Bar.
a. Cells indicating Slow and Deep Breath (FIG. 9a)
Inhalers, that need the user to breathe in uniformly (slowly and deeply), are indicated by equally spaced blocks. The filling of color is also animated uniformly over a period of 3 seconds (this time may change per inhaler). An instance of the same is shown in FIG. 9a.
b. Cells indicating Quick and Deep Breath (FIG. 9b)
Inhalers, that need the user to breathe in quickly at the beginning, are denoted by shorter and tightly spaced blocks that gradually become taller during the deep phase of breathing in. The colors fill in fairly quickly at the initial “Quick” phase and gradually fill in at the top when the breathing needs to be deep. An instance of the same is shown in FIG. 9b.
(iii) Moving Arrow
The vertically moving arrow is secondary visual indication of breathing in or out. Arrow moving towards the top denotes breath in and arrow moving towards the bottom denotes breathing out.
It should be noted that depending on the inhaler the moving arrow may or may not be displayed to reduce visual clutter.

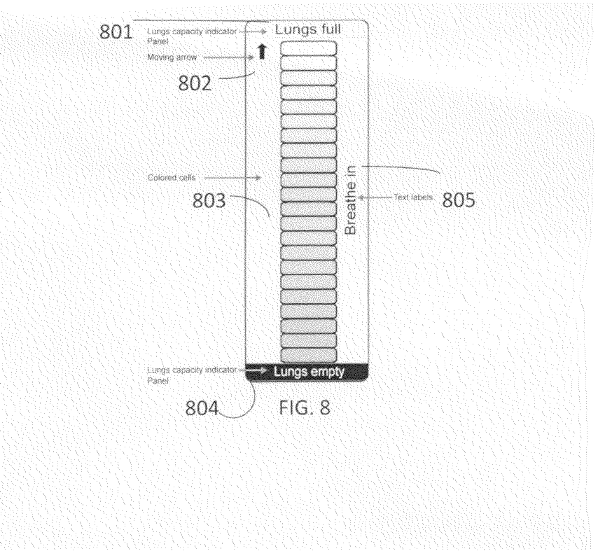

### (iv) The “Press-Inhaler” Indicator (FIG. 10)

This is a special indicator that appears on the Breath Bar for inhalers where the patient needs to actuate or press the inhaler canister or button to release the medicine. The position of the indicator denotes at which point of time the user needs to press the inhaler, while breathing in. When the colored blocks pass through this indicator, the indicator glows bright and a “beep” tone is produced. An instance of an embodiment of the “Press Inhaler” Indicator is shown in FIG. 10.

### (v) Text Labels

Text labels are tertiary indicators to denote breathe in and out. The text Breathe In appears when the breath bar travels up and Breathe Out when the breath bar travels to the bottom. In special cases where the breathing pattern needs to be Quick and Deep, the Text Quick and Deep appears alongside the colored cells.

Audio Component

The Visual Breath Bar is accompanied with an Audio Tone. Different notes of the same tone are played during breathe in and out. The tone has a higher pitch when breathing in and lower pitch when breathing out. The tones are played with gradual increase of volume from bottom to the top and vice versa.

It should be noted that the Audio Tone helps the patient/related health professional to follow the breathing pattern for each inhaler by a sound pattern vis-à-vis the visual component (the Breath Bar animation). The sound is made available even when the Breath Bar disappears from the screen. As the patient remembers the sound pattern, s/he can sync her/his breath with the sound, instead of the Breath Bar animation, even when s/he is not looking at the screen.

Press Inhaler Beep: A generic beep sound is used to indicate pressing of inhaler.
Subsequently, this same sound pattern is used across all Stages.

Timing Component

The whole breath bar is driven by a timing pattern. The timing differs from inhaler to inhaler. For some inhalers, the timing needs to be 3 seconds or breathing loop where others require timings of 3.5 or 4 seconds maximum. The colors within the cells, the moving arrow, glowing of the Lung Capacity indicator, the audio tones, the Press Inhaler indicator and beep are all dependent on the timing of the breath bar.

### Example

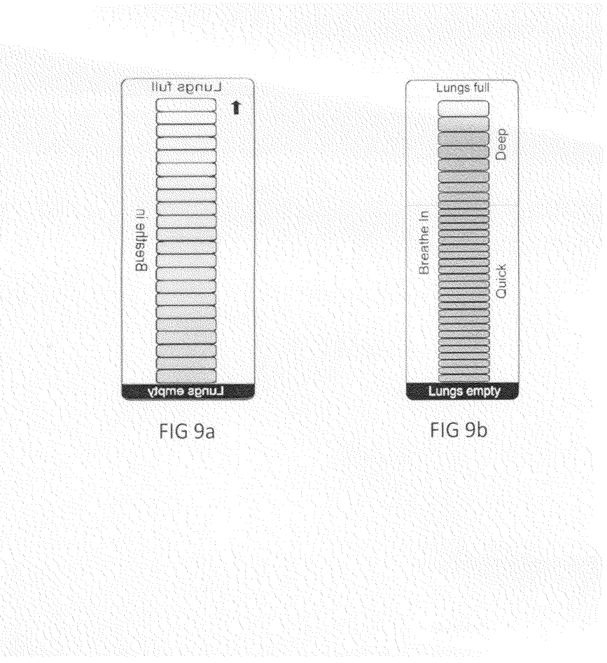

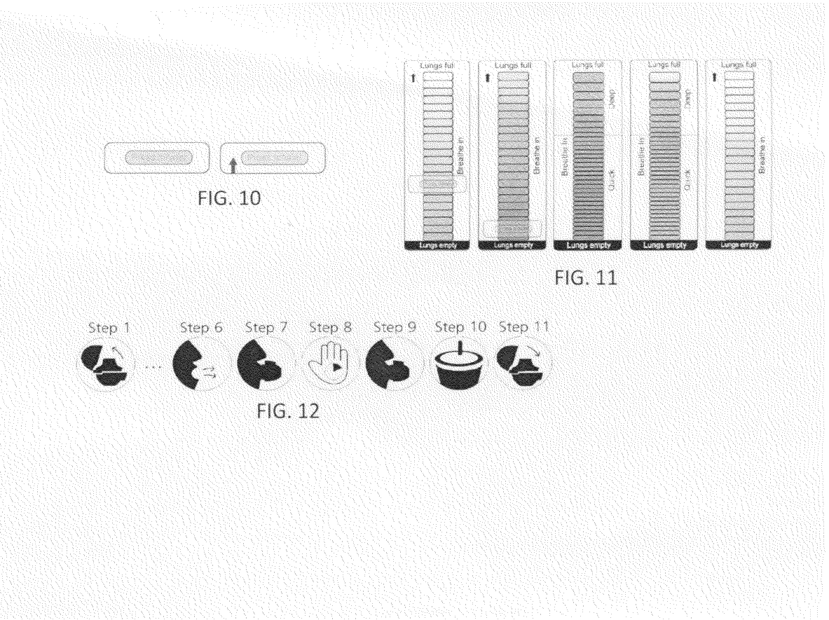

### How the Breath Bar is Used for Different Inhalers (FIG. 11) and Described in Table I

TABLE I

| | | MDI with | | | Breath Actuated |
|---|---|---|---|---|---|
| Category | MDI/Open Mouth | Spacer | Powder Based Inhalers | Capsule Based Inhalers | Inhalers |
| Breath | Example FIG. 11a | Example | Example FIG. 11c | Example FIG. 11d | Example |
| Bars | | FIG. 11b | | | FIG. 11e |
| Inhalers | (i) Metered Dose | Metered | (i) Diskus | (i) Handihaler | (i) Autohaler |
| | Inhaler without | Dose Inhaler | (ii) Twisthaler | (ii) Aerolizer | |
| | Spacer | with | (iii) Turbuhaler | (iii) Neohaler | |
| | (ii) Respimat | Spacer | (iv ) Flexhaler | (iv) Rotahaler | |
| | | | | (v) Octahaler | |
| | | | | (vi) Revolizer | |
| | | | | (vii) Lupihaler | |

In short, the Breath Bar acts as a cue on how to breathe-in and breathe-out with a specific inhaler. To enable the patient further, to use her/his sub-conscious memory every time s/he is learning or using the inhaler, the Breath Bar is accompanied with a unique sound, breath tone audio. This audio is useful even when the patient is not looking at the display and hence, acts as a good memory trigger.
In an enhancement to the guidance a real-time video of the learner displayed in the computing device camera, is superimposed by resultant visual effects (including but not limited to animated or moving images) as a result of proper or improper responses to questions asked by the self-learning program about the usage of the inhaler device.

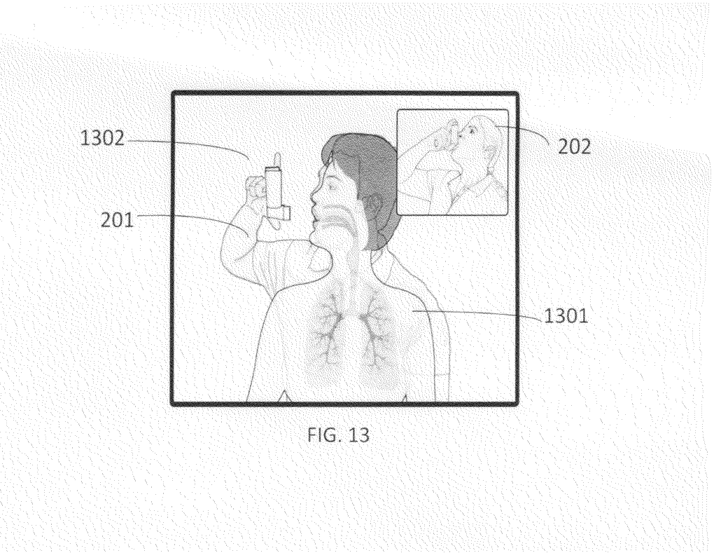

Stage 3, is further enhanced to provide real-time feedback to the patient about the mistakes s/he needs to avoid during actual usage. Similar to the Stage 3, the patient is presented with a list of questions (that represent the Dos and Don'ts while using an inhaler). S/he needs to respond to these questions from an existing list of responses. On selection of a correct or incorrect response, the patient's real-time video (seen through a device camera, where the device is a PC, Smartphone, Tablet or any wearable or implantable computing device) is superimposed with transparent imaging that animates or demonstrates what happens to the internal or otherwise non-visual effects within or outside the patient's anatomy (or body). This superimposition may be accompanied with audio, textual or other instructions to explain the key concepts that may not be comprehensible, otherwise. This superimposition enables learning and explanation of anatomical, non-anatomical changes, atmospheric changes and other changes in the surroundings, depicted in an augmented reality environment. An instance of this is shown in FIG. 13. FIG. 13 shows the real-time simultaneous viewing of the patient's real-time video 201 using an inhaler 1302 along with the instructional video 202 along with a transparent animation 1301 superimposed on the patient's real-time video 201.

In another enhancement to the guidance procedure, a virtual inhaler is used instead of a placebo inhaler. Placebo inhalers are generally used by the providers. Several researches show multipatient placebo inhaler devices is a matter of issue because of the potential risk of cross infection to patients. The current practice is variable and has the potential for poor clinical practice thus placing the patients at risk. Also, the cleaning of the multi patient placebo inhalers may be questionable at times. There are certain placebo inhalers available for one time use only. However, that increases the cost factor for the providers. Thus, by using a virtual inhaler, the cost factor as well as the cross infection to patients is taken care of. By using a virtual inhaler, there is no question of cross-infection to the patients. Also, it saves the cost of buying a placebo inhaler-(both one-time use and multi patient.)

Another aspect of the Guidance/Self-learning process of using inhalers is described below. It talks about the automated reminder feature that allows the patient to use inhalers on a regular/daily basis. It has multiple modes/stages (For example: Real-Time mode, Learn mode, etc)

In this process, in one of the modes, the patient (user/learner) can use the reminder feature to remind him about the time and the dose of the inhaler medication along with listening to audio and/or viewing audio-visuals, if he desires so, to take the correct dose with the correct technique of using the inhaler.

Another mode, for instance, the Learn Mode, provides a unique feature that allows the patient to understand the importance and correct technique of breathing. It also teaches and advices on the importance and timing of processes like priming, cleaning and rinsing along with audio-visuals of the respective processes if the patient desires to see so. This can be followed by an interactive quiz regarding these processes and a Completion Certificate on the successful completion of the quiz.

Another mode, for instance, the Real-Time Mode of the innovation allows the user to set the following features which assist the patient in using the inhaler on a regular/daily basis:
Number of puffs of the inhaler
Time of the day for using the inhaler
Inhaler type to be used by the patient
Dosage and type of inhaler medicine
Reminder by location where the inhaler has to be used

The real-time mode also allows the patient to directly go to the practice session if he desires so. It can assist the patient with an audio and/or audio-visual depending on his choice.

The real-time mode of guidance logs each use of the inhaler used by the patient. Based on this it suggests/reminds/brings up processes like priming and cleaning when required.

Based on the inhaler medication used, the real-time mode brings up/suggests/reminds about rinsing to the patient.

The real-time mode also allows the patient to indicate/log if the inhaler medication was taken without going through the instructions. This can be done by several ways-manually, vibration, voice, etc

It can also send alerts and notifications to parents/caregivers if the inhaler medication was not taken.

The real-time mode has a unique feature of capturing the weather and the spatial location of the patient.

The real-time mode of guidance allows logging and recording of the practice sessions. This can be used later for analysis by patients/parent/caregiver/health-professional.

Thus, the guidance/self-learning process of using inhalers is a complete system wherein a patient can learn, practice and utilize on a regular basis, the correct technique of using inhalers without any exterior intervention. By doing so, he not only learns the correct technique of using inhaler devices but also reinforces them by regular practice. Also, this innovative system makes it possible for the patient to take his doses regularly and on time without having to worry about skipping the doses. Thus, this guidance system as a whole improves the effect of the inhaler medication and hence the overall treatment of the patient.

1. A self-learning system of using inhaler devices comprising of multiple stages of guidance which includes real-time learning using the camera device such that it provides simultaneous viewing of the instructional video and the patient's real-time video, allows the patient to learn and/or practice at his/her own pace, assists the patient for regular use of the inhaler device by, logging, analyzing and alerting about the correct dosage and usage of the inhaler device while allowing an option to record the practice sessions for either self/caretaker analysis or analysis by an expert to get feedback.

2. The self-learning system of using inhaler devices as claimed in claim 1 wherein the plurality of stages further comprises of Real-time Assistance, Guidance of inhalation procedure, Guidance of using the inhaler correctly, Guidance on avoiding most common mistakes via an interactive quiz and recording the real-time video for analysis.

3. The self-learning system of using inhaler devices as claimed in claim 2 further comprising:
a stage for guidance on the need, timing and importance of priming, cleaning and rinsing; and
providing audio-visuals regarding the need, timing and importance of priming, cleaning and rinsing; wherein the timing and amount of these processes are calculated based on type of inhaler device, usage log of the user and inhaler medicine content.

4. The system of inhaler devices as claimed in claim 1 further comprising
reminding about the number of puffs to be taken, type and dosage of the inhaler medicine to be taken, the type of inhaler to be used;
assisting in using the inhaler device correctly by audio/audio-visual/practice session,
setting a reminder for using the inhaler by location and time, capturing weather parameters and spatial location of the patient for analysis; and
alerting a parent or caregiver if the inhaler was not taken.

5. A technique to optimize the breathing pattern of the patient by an Audio-Visual Indicator in conjunction with the guidance procedure such that it assists the patient in syncing his breathing to the said Audio-Visual Indicator and consequently results in optimizing his breathing technique for the chosen inhaler device.

6. A method of self-learning usage of an inhaler device comprising:
defining a plurality of guidance stages connected pedagogically through interactive feedback on a rendering device;
iteratively rendering a sequence of the defined stages and obtaining a feedback result indicative of the success of each stage;
upon completion of each stage, reverting to a previously rendered stage or advancing to a successive stage based on user feedback; and
iteratively advancing through the plurality of stages,
the guidance stages including an inhalation stage, the inhalation stage having simultaneous rendering of a real-time user video stream alongside an instructional video stream depicting correct usage of the inhaler.

7. The method of claim 8 further comprising a demonstration stage, the demonstration stage rendering a video stream depicting a proper inhalation sequence, and preceding the instructional stage in the sequence of stages.

8. The method of claim 9 further comprising:
rendering a breath bar indicative of a volume of air moving through the inhaler;
and animating a visual indicator on the breath bar to indicate an aggregate volume of air moved by the user.

9. The method of claim 8 further comprising:
displaying a progress indicator, the progress indicator having an icon corresponding to each of the stages arranged in a linear manner; and
outlining an icon corresponding to a greatest stage in the sequence completed by the user.

10. The method of claim 11 wherein the stage includes a popup window for directive content relating to completion of the stage.
